(19) United States
(12) Patent Application Publication
(10) Pub. No.: US 2018/0243350 A1
(43) Pub. Date: Aug. 30, 2018

(54) THERAPEUTIC AND PROPHYLACTIC COMPOSITION PRODUCED BY MICROBIOTA

(71) Applicant: Investment SA Neuchatel, Neuchatel (CH)

(72) Inventors: Jan Raa, Oslo (NO); Arnold Berstad, Oslo (NO); Atti-La Hans Ake Ben Dahlgren, Bevaix (CH); Tore Midtvedt, Gjettum (NO)

(73) Assignee: Investment SA Neuchatel, Neuchatel (CH)

(21) Appl. No.: 15/552,182

(22) PCT Filed: Feb. 16, 2016
(86) PCT No.: PCT/SE2016/050119

§ 371 Date: Aug. 18, 2017

(30) Foreign Application Priority Data
Feb. 19, 2015 (SE) 1550189-3

(51) Int. Cl. A61K 35/741; A23L 33/135; C12N 1/02; C12N 1/20; A61P 1/00; A61P 37/06

(52) U.S. Cl. CPC A61K 35/741; A23L 33/135; A61P 37/06; C12N 1/20; A61P 1/00; C12N 1/02

(57) ABSTRACT

The invention regards a biochemical composition obtained after separating microbes and solid, fibrous matter from a microbial ecosystem (=microbiota) by removing the microorganisms from a growth medium in which intestinal microbiota and/or faeces have been anaerobically cultivated and freeing the medium from the microbiota and the use thereof as a pharmaceutical and for the treatment or prophylaxis of diseases.

### FIELD OF INVENTION

The invention regards a biochemical composition obtained after separating microbes and solid (e.g. fibrous) matter from a microbial ecosystem (=microbiota) grown on microbial growth media and the use thereof for the treatment or prophylaxis of diseases.

The cultured microbiota used in this invention may be sourced from any multi-cellular animal, including mammals and humans.

### PRIOR ART

The gastrointestinal tract contains several complex micro-ecological systems, in which the microbes interact and “communicate” mutually and with the host organism by interactions with gut epithelia.

The natural ecological balance between microbes constituting the gut microbiota can be disturbed by external and internal physical, chemical and biological factors or agents, the consequence of which can be severe infections and/or disturbed metabolic and organ functions.

Infections by Clostridium difficile is an example of a severe bacterial infection causing life-threatening diarrhoea if the ecological balance of the gut microbiota is disrupted. Clostridium difficile associated diarrhoea (CDAD) was first described around ⅓ of a century ago. In spite of thousands of publications in PubMed of the pathogenesis, epidemiology, clinical diagnosis and various therapeutic approaches, CDAD continues to persist as a costly, leading cause of infectious health-care-associated gastrointestinal illness, and the number of incidences is rising sharply. A tenfold increase of CDAD has occurred in Europe, USA and Canada in the last 10 years.

Increasing therapeutic failures of metronidazole and vancomycin treatments have been reported. New antibiotics, such as fidaxomicin, oritavancin, nitazoxanide, REP3123 and NVB303, are on their way to the market to cope with this alarming disease problem. It has to be recognized that even before these drugs are used, a certain number of Clostridium difficile bacteria have reduced sensitivity to new antibiotics. As a consequence these bacteria will become the dominant ones in environments containing the new antibiotics, and the antibiotics will inevitably and gradually lose their efficacy. Additionally, whatever their clinical efficacy may turn out to be, they will certainly be expensive in use.

Many CDAD-patients experience relapsing diarrhoea after completed and repeated antibiotics treatment. Other treatment strategies have therefore been in focus for some years. The most effective treatment, called Faecal Microbiota Transplantation (FMT), involves introducing a beneficial gut microbiota by transplantation of fresh faeces from a healthy donor, mostly a close relative.

FMT is usually carried out by gastrointestinal infusion of liquid faeces suspensions. Microbiota transplantation has also been carried out by oral intake of encapsulated faeces preparations, for use in the treatment of gastrointestinal disorders.

FMT is not without risk. A main problem is the risk of transmission of bacteria carrying genes resistant to antibiotics. The transfer of such genes may have serious consequences for the patient when antibiotic treatment is needed in the future. Another main problem with FMT is the risk of transmission of potentially contagious agents present in the donor's faeces. To reduce this risk the donors have to undergo careful health screening and the faeces samples must be subjected to comprehensive, time-consuming and expensive laboratory testing. This type of treatment is further perceived by many people, both doctors and patients, as highly unaesthetically.

Below is a selection of publications describing the prior art of FMT:
Johan S. Bakken. Faecal bacteriotherapy for recurrent Clostridium difficile infection. Anaerobe 15 (2009) 285-289.
van Nood E, Vrieze A, Nieuwdorp M, et al. Duodenal infusion of donor feces for recurrent Clostridium difficile. N Engl J Med. 2013;368(5):407-415.
Austin M, Mellow M, Tierney W M. Fecal microbiota transplantation in the treatment of Clostridium difficile infections. Am J Med. 2014;127(6):479-483.
Youngster I, Sauk J, Pindar C, et al. Fecal microbiota transplant for relapsing Clostridium difficile infection using a frozen inoculum from unrelated donors: a randomized, open-label, controlled pilot study. Clin Infect Dis. 2014;58(11):1515-1522.

In all literature on FMT there is a common and generally accepted explanation of the therapeutic efficacy of this treatment, namely restored ecological balance of the patient's gut microbiota by re-introduction of live gut bacteria which of various reasons have been outnumbered.

It has been found that also live intestinal microbiota grown artificially in laboratory culture, has therapeutic effects comparable to those of FMT. This has been described in the following publications:
C. Jorup-Ronstrom, A Hakansson, A-K Person,T Midtvedt, E Norin. Feceskultur framgangsrik terapi vid Clostridium difficile-diarre. Lakartidningen nr 46 2006 volym 103, 3603-3605.
A. Gustafsson, A. Berstad, S. Lund-TØnnesen, T. Midtvedt, E. Norin. The Effect of Faecal Enema on Five Microflora-Associated Characteristics in Patients with Antibiotic-Associated Diarrhoea. Scand J Gastroenterol. 1999;34:580-586.

A. Gustaysson, S. Lund-TØnnesen, A. Berstad, T. Midtvedt & E. Norin Faecal Short-Chain Fatty Acids in Patients with Antibiotic-Associated Diarrhoea, before and after Faecal Enema Treatment. Scand J Gastroenterol 1998;33:721 -727.

As prior art should also be included WO 2011/033310. This invention relates to an anaerobic micro-ecological system comprising anaerobically cultivated human intestinal microbiota and the use thereof as a functional seeding culture for re-establishing normality of a disturbed human microbiome and gastrointestinal functions. It also regards the use of the composition for the prevention and treatment of disease and a method for preparing the composition.

The therapeutic efficacy of transplanting artificially cultivated microbiota is being ascribed to the live transplanted bacteria, as for FMT.

It should be noted that the therapeutic potentials of FMT and of transplantation of artificially grown intestinal bacteria according to WO 2011/033310, go far beyond CDAD. Disturbed gut microbiota is the root-cause of a number of diseases affecting millions of people worldwide, predominantly in Western type societies. Diseases such as diabetes (type 1 and type 2), metabolic syndrome, morbid obesity, irritable bowel syndrome (IBS), inflammatory bowel diseases (Crohn's disease, ulcerative colitis), neurological and behavioral disorders (chronic fatigue syndrome, autism spectrum disorders, ADHD, Parkinson's disease), inflammatory and immunological disorders (asthma and allergy) and antibiotic associated diarrheas (AAD), are all associated with ecological imbalances in the gut microbiota.

### SUMMARY OF THE INVENTION

It has now been found that a composition prepared from the medium in which intestinal microbiota and/or faeces have been growing, and from which live bacteria have been removed, can be used for the treatment or prophylaxis of diseases, and for immediate eleviating of pain of patients sufferings from gastrointestinal infections.

One advantage of the present invention is that no bacteria are transferred from a donor as in the case of FMT which dramatically reduces the risk of transferring bacterial genes that may have a negative influence on the health of the human or animal recipient.

The therapeutic implications of this highly unexpected discovery have been demonstrated in a number of disorders, for instance in human patients who were severely ill from irritable bowel syndrome (IBS) and chronic fatigue syndrome/ME. After infusing the composition of the present invention via gastroscope into distal duodenum, the patients quickly experienced relief from stomach pain and notably reduced diarrhoea, and got firmer stool.

The composition of the present invention contains a mixture of components produced by all microbes of the complete microbiota during their growth on artificial growth media. These microbiota-derived components, which are present also inside the colon of healthy humans and animals, may include fatty acids, hydrogen sulphide, ammonia and many different volatile and non-volatile fermentation products. Although some of these components individually may affect gut functions, none of them have ever been shown to have any therapeutic potential, neither alone or in predetermined combinations with other specific fermentation products.

As opposed to prior art on health effects of specific fermentation products or combination of such products produced by intestinal bacteria, the present invention regards a composition which by concerted action of the components it contains, provide therapeutic and prophylactic efficacy.

It follows as a logical consequence of the present invention that the Composition described herein can be used in conjunction with, and combined with, many other substances or agents with the purpose to improve effects and practical usage. The Composition is a biochemical package of all product made by concerted action and interaction of a great number of microbes constituting a complex ecosystem of intestinal microbiota. Hence, the Composition may be used as a stand-alone product to improve gut health, and as an prebiotics preparation that contribute to stabilizing the ecological interaction of a microbiota introduced as FMT or as cultivated intestinal microbiota.

### DETAILED DESCRIPTION OF THE INVENTION

In the present application the term “microbiota” is used for the microorganisms that typically inhabit the gastrointestinal system.

The invention regards a composition obtained by removing the microorganisms from a growth medium in which intestinal microbiota has been cultivated in a physiologically acceptable medium and freeing the medium from its microbiota content.

A first aspect of the present invention is a composition comprising a supernatant obtained from cultivation of intestinal microbiota in a cultivation medium and deletion of the microbiota by centrifugation or filtration.

According to one embodiment the spent growth medium according to the present invention is the culture filtrate collected after growth of a complete microbial ecosystem on an artificial laboratory medium, according to WO2013/053836 A1, which is hereby incorporated as reference.

A complete ecosystem of hundreds of different bacterial species may be produced under anaerobic conditions, to maintain the microbial diversity. Strict anaerobic conditions may be used.

The intention of using this bacteria-free—non-feces preparation—is not to reinstall missing microbes, the very purpose of FMT (=fecal microbiota transplantation), but rather to use the growth medium isolated and separated from the bacteria.

Quite surprisingly, and unexpectedly, it was found that the multitude of “Eco stabilizing” products remaining after anaerobic growth of a great number of bacterial species in ecological stability, have several clinical effects against e.g. against IBS as described in Example 4.

According to one embodiment a slurry of faeces is made in a physiologically acceptable medium, e.g. in physiological saline. This may be done by mincing the faeces e.g. by a mixer in the medium. The medium may be freed from the faeces material by filtration or by obtaining a supernatant after centrifugation after contact therewith for up to four days, e.g. for 1-3 days, 2-14 hours, 1-60 minutes such as 10-60 minutes. The temperature may be between 15 and 40 ° C., preferably room temperature. The whole procedure may be done under strict anaerobic conditions or not.

The intestinal microbiota used in the invention may derive from any part of the gastrointestinal system of any animal, such as e.g. cattle, horse, cat, dog, sheep, goat, chicken, duck, goose. According to one embodiment the intestinal microbiota are from any part of the human gastrointestinal system, e.g. stomach, duodenum, small intestine, cecum or colon.

The faeces and/or the microbiota may derive from any species and the composition or supernatant freed from solid material and cells may be given to any species. According to one embodiment the faeces and/or microbiota derive from—and the composition or supernatant is given to—the same species.

The microbiota may be cultivated on bacterial growth media, preferably anaerobically, but also in the presence of sufficient oxygen and other metabolic electron acceptors to allow growth of facultative anaerobic and microaerophilic microorganisms.

The medium may be freed from the microbiota after contact therewith for up to four days, e.g. for 1-3 days, 2-14 hours, 1-60 minutes such as 10-60 minutes. The temperature may be between 15 and 40° C., preferably room temperature.

According to one aspect of the present invention anaerobically cultivated human intestinal microbiota is used to produce the composition of the present invention. According to one embodiment the microbiota used in this cultivation contains bacteria belonging to at least three of the following four phyla: Bacterioidetes, Firmicutes, Proteobacteria and Actinobacteria.

The cultivation medium may be a yeast based peptone comprising added cholesterol, preferably cholesterol of animal origin, and freeze dried hen yolk in an amount of from 0.5 to 5%, preferably 1.25% w/v (weight per volume).

According to one embodiment a pre-reduced sterilized bacteriological peptone yeast based medium (Difco, USA) is used with addition of 1.25% (w/v) freeze dried hen yolk (Fresenius-Kabi Sweden) with resazurin as an anaerobic indicator.

According to another embodiment a peptone-yeast medium (Difco, USA), containing cholesterol of animal origin and 1.25% freeze-dried hen yolk (Fresenius-Kabi, Sweden), with resazurin as an anaerobic indicator, is used.

The composition of the invention contains products produced during growth of bacteria belonging to the phyla mentioned above. This composition, from which bacteria have been removed, has surprisingly shown to be useful in the treatment or prophylaxis of disease.

Diseases that can be treated or prevented by the invention are for example diarrhoeas following antibiotic treatment (Antibiotic Associated Diarrhoeas), especially diarrhoeas caused by Clostridium difficile which can be very long lasting and difficult to treat by conventional methods and can in rare cases lead to death. Other therapeutic areas are Irritable Bowel Syndrome (IBS), Celiac Disease and Inflammatory Bowel Diseases for example Ulcerative Colitis, Crohn's Disease, Microscopic Colitis, and Pauchitis. Another treatment areas are iatrogenic disturbance/dysbiosis of the intestinal microbiome, e.g. following radiation therapy, chemotherapy and in connection with transplantations. Examples of other diseases that can be treated or prevented with the invention are: neurological diseases for example Parkinson's Disease, Alzheimer's Disease, Lou Gehrig's Disease ALS—Amyotrophic Lateral Sclerosis, Multiple Sclerosis; behavioral/psychiatric disorders e.g. Autism, Asperger's Syndrome , Attention Deficit Hyperactivity Disorder (ADHD) and Depression; Rheumatologic diseases e.g. Rheumatoid arthritis; and systemic/metabolic disorders for example: Hypertension, Obesitas and type-2 Diabetes, and neoplastic/tumour diseases of the gastrointestinal tract. The invention can also be used in the treatment of Chronic Fatigue Syndrome. The invention can also be used in the treatment of dermatological diseases such as acne vulgaris.

Filtrate obtained from cultivated microbiota from animals or humans, from birth to the time of the establishment of an adult microbiota, may be used to treat and prevent disorders that have its origin in the lack of the right composition of the microbiota or its filtrate in the first years of development. Examples of such disorders are: autism, autism spectrum disorders, myopia, diabetes type 1, allergies, atopic eczema and asthma.

In addition to humans, the composition of the invention may be administrated to any animal such as domestic animals e.g. cattle, pigs, horse, cat, dog, sheep, goat, chicken, duck, goose and animals in aquaculture, e.g. fish and shrimp.

The composition according to the present invention may be administrated to animals and humans by a device selected from a naso-duodenal tube, gastroscope, colon/sigmoideoscope, and enema, or in freeze dried form in a suitable galenic preparation, e.g. gastric acid resistant capsule, nano-encapsulation or suppository.

In one embodiment the composition may also contain other added agents or factors, including carrier materials and signaling substances active against diseases.

The composition may be provided as: a) Freshly prepared liquid culture filtrate or centrifugation supernatant; b) Frozen preparation to be thawed prior to administration to patients; c) Dried or freeze dried liquid culture filtrate or centrifugation supernatant; d) Capsules containing liquid or dried product: e) Tablets and granulates.

The daily dosage should be adjusted to an amount of active material corresponding to the content of 1-100 ml of the freshly prepared composition, preferably 20-40 ml.

The invention may be applied into the gastro-intestinal tract at least one time per day during the course of a number of days in a year, e.g. 1 -365 days, depending on the nature, cause and severity of the problem.

The product can preferably be administered according to the following non limiting examples: in the form of a solution through e.g. a naso-duodenal tube, gastroscope, colon/sigmoideoscope, enema or in freeze dried form in a suitable galenic preparation, e.g. a gastric acid resistant capsule, nano-encapsulated or a suppository.

Other substances and components which facilitate the administration or supports the effect of the invention may be added to the composition, for example β-glucans, thylakoids, bacterial, and algae preparations.

The composition may comprise pharmaceutically-acceptable excipients. The term “pharmaceutically-acceptable excipients”, as used herein, includes any physiologically inert, pharmacologically inactive material known to one skilled in the art, which is compatible with the physical and chemical characteristics of the composition. Pharmaceutically-acceptable excipients include, but are not limited to, solvents, co-solvents, buffer systems, surfactants, preservatives, sweetening agents, flavoring agents, pharmaceutical grade dyes or pigments, viscosity agents, a isotonizing agent, a soothing agent, a preservative, an antioxidant, and a colorant.

The Composition contains a mixture of products made by concerted action of microbes constituting a complex ecosystem of intestinal microbiota. The Composition has therefore logical applications within the prebiotics sector, and as a preparation which pre-conditions the biochemical environment in the digestive system for successful establishment of intestinal microbiota introduced by FMT or by artificially cultivated microbiota. The Composition may also be used in dietary formulations for animals and humans.

The invention will now be described by the following non-limiting examples.

### EXAMPLES

### Example 1: Cultivation of the Microbiota

Microbiota according to the present invention were obtained from a stool sample from a healthy single donor prior to 1995. Both the donor and the stool were thoroughly examined. The functional status of the donor's intestinal Microbiota were found to have normal functional values of coprostanol, urobilins, mucin, fecal 30 tryptic activity, short chain fatty acids and beta-aspartylglycine. The tests for hepatitis viruses A, B and C, cytomegalovirus, Epstein-Barr virus, human immunodeficiency virus (HIV), Calici- and Rotavirus were all negative.

Furthermore, the faeces was screened for presence of Salmonella, Shigella, Campylobacter, Yersinia, Clostridium difficile and protozoan cysts, and all these investigations were negative.

A sample of faeces from the interior of the stool sample (Bristol scale type 2) was taken from the above-mentioned healthy donor and immediately suspended in 30 ml of a pre-reduced sterilized bacteriological peptone yeast based medium (Difco, USA), with addition of 1.25% (w/v) freeze dried hen yolk, Fresenius-Kabi Sweden with resazurin as an anaerobic indicator. This composition was re-cultivated under nitrogen flow every second week. The bacterial content of the cultivated product were found to be >10⁹ per ml medium. After addition of 10% glycerol the composition can be stored at −80° C.

The product contained the following species:
Bacteria; “Actinobacteria”; Actinobacteria; Coriobacteridae; Coriobacteriales; “Coriobacterineae”; Coriobacteriaceae; Collinsella,
Bacteria; “Actinobacteria”; Actinobacteria; Coriobacteridae; Coriobacteriales; “Coriobacterineae”; Coriobacteriaceae; Eggerthella,
Bacteria; “Actinobacteria”; Actinobacteria; Coriobacteridae; Coriobacteriales; “Coriobacterineae”; Coriobacteriaceae; Olsenella,
Bacteria; “Bacteroidetes”; “Bacteroidia”; “Bacteroidales”; “Porphyromonadaceae”; Parabacteroides,
Bacteria; “Bacteroidetes”; “Bacteroidia”; “Bacteroidales”; “Prevotellaceae”; Prevotella,
Bacteria; “Bacteroidetes”; “Bacteroidia”; “Bacteroidales”; “Rikenellaceae”; Alistipes,
Bacteria; “Bacteroidetes”; “Bacteroidia”; “Bacteroidales”; Bacteroidaceae; Bacteroides
Bacteria; “Firmicutes”; “Bacilli”; “Lactobacillales”; “Enterococcaceae”; Enterococcus,
Bacteria; “Firmicutes”; “Clostridia”; Clostridiales; “Eubacteriaceae”; Eubacterium
Bacteria; “Firmicutes”; “Clostridia”; Clostridiales; “Eubacteriaceae”; unclassified_“Eubacteriaceae”,
Bacteria; “Firmicutes”; “Clostridia”; Clostridiales; “Lachnospiraceae”; Anaerostipes,
Bacteria; “Firmicutes”; “Clostridia”; Clostridiales; “Lachnospiraceae”; Dorea,
Bacteria; “Firmicutes”; “Clostridia”; Clostridiales; “Lachnospiraceae”; Roseburia,
Bacteria; “Firmicutes”; “Clostridia”; Clostridiales; “Lachnospiraceae”; unclassified_“Lachnospiraceae”,
Bacteria; “Firmicutes”; “Clostridia”; Clostridiales; “Peptostreptococcaceae”; Sporacetigenium,
Bacteria; “Firmicutes”; “Clostridia”; Clostridiales; “Peptostreptococcaceae”; unclassified_“Peptostreptococcaceae”,
Bacteria; “Firmicutes”; “Clostridia”; Clostridiales; “Ruminococcaceae”; Anaerofilum,
Bacteria; “Firmicutes”; “Clostridia”; Clostridiales; “Ruminococcaceae”; Anaerotruncus,
Bacteria; “Firmicutes”; “Clostridia”; Clostridiales; “Ruminococcaceae”; Oscillibacter,
Bacteria; “Firmicutes”; “Clostridia”; Clostridiales; “Ruminococcaceae”; unclassified_“Rurninococcaceae”,
Bacteria; “Firmicutes”; “Clostridia”; Clostridiales; Clostridiaceae; “Clostridiaceae1”; Clostridium,
Bacteria; “Firmicutes”; “Clostridia”; Clostridiales; Clostridiaceae; “Clostridiaceae1”; Sarcina,
Bacteria; “Firmicutes”; “Clostridia”; Clostridiales; Clostridiaceae;“Clostridiaceae1”; unclassified_“Clostridiaceae1”,
Bacteria; “Firmicutes”; “Clostridia”; Clostridiales; IncertaeSedisXI; Anaerococcus,
Bacteria; “Firmicutes”; “Clostridia”; Clostridiales; IncertaeSedisXI; Finegoldia,
Bacteria; “Firmicutes”; “Clostridia”; Clostridiales; IncertaeSedisXI; Peptoniphilus,
Bacteria; “Firmicutes”; “Clostridia”; Clostridiales; IncertaeSedisXI; unclassified_IncertaeSedisXI,
Bacteria; “Firmicutes”; “Clostridia”; Clostridiales; IncertaeSedisXIII; Anaerovorax,
Bacteria; “Firmicutes”; “Clostridia”; Clostridiales; IncertaeSedisXIII; Mogibacterium,
Bacteria; “Firmicutes”; “Clostridia”; Clostridiales; IncertaeSedisXIV; Blautia,
Bacteria; “Firmicutes”; “Clostridia”; Clostridiales; unclassified_Clostridiales,
Bacteria; “Firmicutes”; “Clostridia”; Clostridiales; Veillonellaceae; Veillonella,
Bacteria; “Firmicutes”; “Erysipelotrichi”; “Erysipelotrichales”; Erysipelotrichaceae; Holdemania, and
Bacteria; “Proteobacteria”; Betaproteobacteria; Burkholderiales; Alcaligenaceae; Parasutterella,

After addition of 10% glycerol the composition can be stored at −80° C.

### Example 2. Preparation of Composition by Removing Bacteria and Fibrous Matter from Microbiota Culture

Centrifugal tubes containing the cultivated microbiota produced according to Example 1, were subjected to centrifugation for 30 minutes at a rotation speed corresponding to a G-force of 1000. A foamy surface layer of lipid-like material was sucked off, the liquid interphase decanted and then re-centrifuged for another 30 minutes at 1000 G to sediment remaining bacteria and fibrous matter. The decanted liquid was then subjected to ultrafiltration through a 0.25 micron micro filter to ensure sterility of the Composition.

### Example 3: Cultivation of Poultry Microbiota

Fresh faeces from a healthy laying hen living under ecological conditions at a traditional small-scale poultry farm were used as source for anaerobic cultivation of poultry microbiota under conditions comparable as described in Example 1, except that cultivation temperature was 41 C. Under these anaerobic growth conditions the yield of bacteria was between 10⁹ and 10¹⁰ after 7 days of cultivation. The filtrate according to the invention was given to newly hatched chickens in order to facilitate the colonization of a healthy microbioal gut flora.

### Example 4: Clinical Efficacy

On Jan. 19, 2015 a 36 years old lady was treated at a Norwegian hospital with the composition of the present invention, prepared as in Example 2. She had for years been suffering from irritable bowel syndrome, musculoskeletal pain and fatigue. At the time of treatment she had strong abdominal pain and a score on chronic fatigue consistent with ME-diagnosis grade I. The composition of the present invention was administrated by means of gastroscope as a 20 ml liquid solution distally in the duodenum of the patient. After 8 days the patient reported that her abdominal pain disappeared almost immediately after the intervention and that the pain free situation lasted for at least 3 days. She also reported that she had less diarrhoea than prior to the treatment.

1.-24. (canceled)

25. A composition for use in the treatment or prophylaxis of diseases in humans and animals, obtained by removing the microorganisms from a growth medium in which intestinal microbiota and/or faeces have been anaerobically cultivated in a physiologically acceptable medium and freeing the medium from the microorganisms,
wherein the cultivated bacteria belong to at least three of the following four phyla: Bacterioidetes, Firmicutes, Proteobacteria and Actinobacteria, said microorganisms are identified as:
Bacteria; “Actinobacteria”; Actinobacteria; Coriobacteridae; Coriobacteriales; “Coriobacterineae”; Coriobacteriaceae; Collinsella,
Bacteria; “Actinobacteria”; Actinobacteria; Coriobacteridae; Coriobacteriales; “Coriobacterineae”; Coriobacteriaceae; Eggerthella,
Bacteria; “Actinobacteria”; Actinobacteria; Coriobacteridae; Coriobacteriales; “Coriobacterineae”; Coriobacteriaceae; Olsenella,
Bacteria; “Bacteroidetes”; “Bacteroidia”; “Bacteroidales”; “Porphyromonadaceae”; Parabacteroides,
Bacteria; “Bacteroidetes”; “Bacteroidia”; “Bacteroidales”; “Prevotellaceae”; Prevotella,
Bacteria; “Bacteroidetes”; “Bacteroidia”; “Bacteroidales”; “Rikenellaceae”; Alistipes,
Bacteria; “Bacteroidetes”; “Bacteroidia”; “Bacteroidales”; Bacteroidaceae; Bacteroides
Bacteria; “Firmicutes”; “Bacilli”; “Lactobacillales”; “Enterococcaceae”; Enterococcus,
Bacteria; “Firmicutes”; “Clostridia”; Clostridiales; “Eubacteriaceae”; Eubacterium
Bacteria; “Firmicutes”; “Clostridia”; Clostridiales; “Eubacteriaceae”; unclassified_“Eubacteriaceae”,
Bacteria; “Firmicutes”; “Clostridia”; Clostridiales; “Lachnospiraceae”; Anaerostipes,
Bacteria; “Firmicutes”; “Clostridia”; Clostridiales; “Lachnospiraceae”; Dorea,
Bacteria; “Firmicutes”; “Clostridia”; Clostridiales; “Lachnospiraceae”; Roseburia,
Bacteria; “Firmicutes”; “Clostridia”; Clostridiales; “Lachnospiraceae”; unclassified_“Lachnospiraceae”,
Bacteria; “Firmicutes”; “Clostridia”; Clostridiales; “Peptostreptococcaceae”; Sporacetigenium,
Bacteria; “Firmicutes”; “Clostridia”; Clostridiales; “Peptostreptococcaceae”; unclassified_“Peptostreptococcaceae”,
Bacteria; “Firmicutes”; “Clostridia”; Clostridiales; “Ruminococcaceae”; Anaerofilum,
Bacteria; “Firmicutes”; “Clostridia”; Clostridiales; “Ruminococcaceae”; Anaerotruncus,
Bacteria; “Firmicutes”; “Clostridia”; Clostridiales; “Ruminococcaceae”; Oscillibacter,
Bacteria; “Firmicutes”; “Clostridia”; Clostridiales; “Ruminococcaceae”; unclassified_“Ruminococcaceae”,
Bacteria; “Firmicutes”; “Clostridia”; Clostridiales; Clostridiaceae; “Clostridiaceae1”; Clostridium,
Bacteria; “Firmicutes”; “Clostridia”; Clostridiales; Clostridiaceae; “Clostridiaceae1”; Sarcina,
Bacteria; “Firmicutes”; “Clostridia”; Clostridiales; Clostridiaceae; “Clostridiaceae1”; unclassified_“Clostridiaceae1”,
Bacteria; “Firmicutes”; “Clostridia”; Clostridiales; IncertaeSedisXI; Anaerococcus,
Bacteria; “Firmicutes”; “Clostridia”; Clostridiales; IncertaeSedisXI; Finegoldia,
Bacteria; “Firmicutes”; “Clostridia”; Clostridiales; IncertaeSedisXI; Peptoniphilus,
Bacteria; “Firmicutes”; “Clostridia”; Clostridiales; IncertaeSedisXI; unclassified_IncertaeSedisXI,
Bacteria; “Firmicutes”; “Clostridia”; Clostridiales; IncertaeSedisXIII; Anaerovorax,
Bacteria; “Firmicutes”; “Clostridia”; Clostridiales; IncertaeSedisXIII; Mogibacterium,
Bacteria; “Firmicutes”; “Clostridia”; Clostridiales; IncertaeSedisXIV; Blautia,
Bacteria; “Firmicutes”; “Clostridia”; Clostridiales; unclassified_Clostridiales,
Bacteria; “Firmicutes”; “Clostridia”; Clostridiales; Veillonellaceae; Veillonella,
Bacteria; “Firmicutes”; “Erysipelotrichi”; “Erysipelotrichales”; Erysipelotrichaceae; Holdemania, and
Bacteria; “Proteobacteria”; Betaproteobacteria; Burkholderiales; Alcaligenaceae; Parasutterella;
wherein said growth medium is:
a physiologically acceptable medium, e.g. a physiological saline;
wherein
the microbiota is cultivated on bacterial growth media, preferably anaerobically, but also in the presence of sufficient oxygen and other metabolic electron acceptors to allow growth of facultative anaerobic and microaerophilic microorganisms.

26. The composition for use according to claim 25, wherein the intestinal microbiota and/or the faeces were selected from humans.

27. The composition for use according to claim 25, wherein the intestinal microbiota and/or the faeces were selected from animals.

28. The composition for use according to claim 25, comprising a filtrate or a supernatant of a physiologically acceptable medium having been in contact with faeces.

29. The composition for use according to claim 25, characterised in that it further contains substances and components which facilitates the administration or supports the effect of the invention,
said substances and components are selected from β-glucans and/or thylakoids, bacterial, and algae preparations.

30. The composition for use according to claim 25, for use as a pharmaceutical.

31. The composition for use according to claim 30, wherein it is used for treatment or prophylaxis of diseases in humans and animals.

32. The composition for use according to claim 30, for use in the treatment of gastrointestinal pain.

33. The composition for use according to claim 30, wherein the composition is used in the treatment of IBS and Chronic Fatigue Syndrome/Myalgic Encephalomyelitis.

34. The composition for use according to claim 30, wherein the composition is used in the treatment of diseases located in the gastrointestinal system, namely Irritable Bowel Syndrome (IBS), Celiac Disease and Inflammatory Bowel Diseases, e.g. Ulcerative Colitis, Crohn's Disease, Microscopic Colitis and Pauchitis, and also iatrogenic disturbance/dysbiosis in connection with radiotherapy, Chemotherapy, transplantations or antibiotic related diarrhoea, especially related to Clostridium difficile (Clostridium difficile associated diarrhoea and recurrent Clostridium difficile infections).

35. The composition for use according to claim 30, wherein the composition is used in the treatment of rheumatologic disorders, e.g. rheumatoid arthritis.

36. The composition for use according to claim 30, wherein the composition is used in the treatment of disorders and diseases caused by dysbiosis in children from birth to the time when an adult type microbiota has been established.

37. The composition for use according to claim 25, wherein the composition is used as a food, medical food, dietary supplement or food additive.

38. The composition for according to claim 25 for use in administration to a human or animal by a device selected from a naso-duodenal tube, gastroscope, colon/sigmoideoscope, and enema, or in freeze dried form in a suitable galenic preparation, e.g. gastric acid resistant capsule, nano-encapsulated or suppository.
